(19) United States
(12) Patent Application Publication
(10) Pub. No.: US 2011/0208131 A1
(43) Pub. Date: Aug. 25, 2011

(54) Universal disposable syringe for vaginal feminine hygiene with hose

(76) Inventor: Liberty Bernardino Uy, Daly City, CA (US)

(21) Appl. No.: 12/803,412

(22) Filed: Jun. 25, 2010

Related U.S. Application Data
(60) Provisional application No. 61/338,903, filed on Feb. 25, 2010.

(51) Int. Cl. A61M 3/02

(52) U.S. Cl. 604/256; 604/257

(57) ABSTRACT

A method and apparatus for the cleansing of the vagina or anus using disposable syringe with hose that has a cap closure assembly that communicates to the openings of any standard, common bottled water that is available in the market.

### CROSS REFERENCE TO RELATED APPLICATIONS

This application claims the benefit of the provisional patent application Ser. No. 61/338,903, filed 2010, Feb. 25 by Liberty B. Uy

### BACKGROUND OF THE PRESENT INVENTION

Vagina irrigation devices have gained wide use in cleansing and treating the female organs. One such system previously known reusable douche have been particularly inconvenient for the user when away from home due to the bulkiness of the equipment and the maintenance of cleaning the device due to the frequent lack of facilities necessary to perform these operations, especially when traveling.

Disposable douches are also inconvenient to use due to its bulkiness and the need to assemble many parts during use.

In addition to the disadvantages of previous inventions, most disposable pre-mix douches are not allowed on airplanes because of the evolving security threats to aviation. It is also too bulky to put inside the purse.

### SUMMARY

This invention may be summarized at least in part with reference to its objective. It is the primary objective of the present invention to provide the most convenient, effective, sanitary, safe to carry, and handy way to douche conveniently without the bulkiness of the kit. All you need to carry is the present invention. When you are ready to use it, just connect it to any standard, common bottled water or any common bottle that has the same dimension.

The present invention can be manufactured as a soft flexible material to allow the user to adjust the syringe to the most comfortable position to avoid discomfort or injury. Therefore, it doesn't limit the manufacturing material to plastic.

Also, this invention can simply be produced, providing inexpensive syringes for healthy vaginal hygiene.

### DRAWINGS-FIGURES

FIG. 1 Is the Perspective View of a syringe for vaginal irrigation in accordance with the preferred embodiment of the present invention. It shows combined Syringe for fitting on bottled water, showing my new design. The bottled water illustrated as broken lines is not part of the claimed design.

FIG. 2 The Side View of the syringe of FIG. 1

FIG. 3 The Top View of the syringe of FIG. 1

FIG. 4 The 3D View of the syringe of FIG. 1

FIG. 5 The Exploded View of the syringe of FIG. 1

### DRAWINGS-REFERENCE NUMERALS

1 Ejection holes

2 Syringe

3 Hose

4 Cap

5 Neck of Common, Standard bottled water

6 Standard, common bottled water

### DETAILED DESCRIPTION OF THE INVENTION

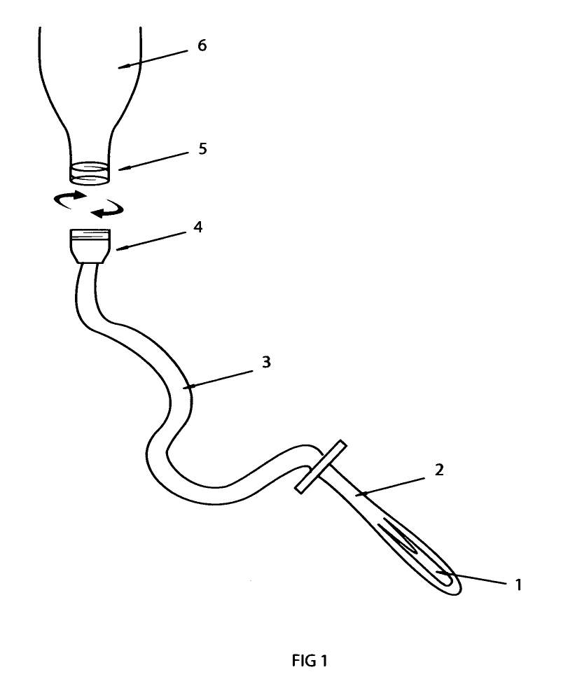

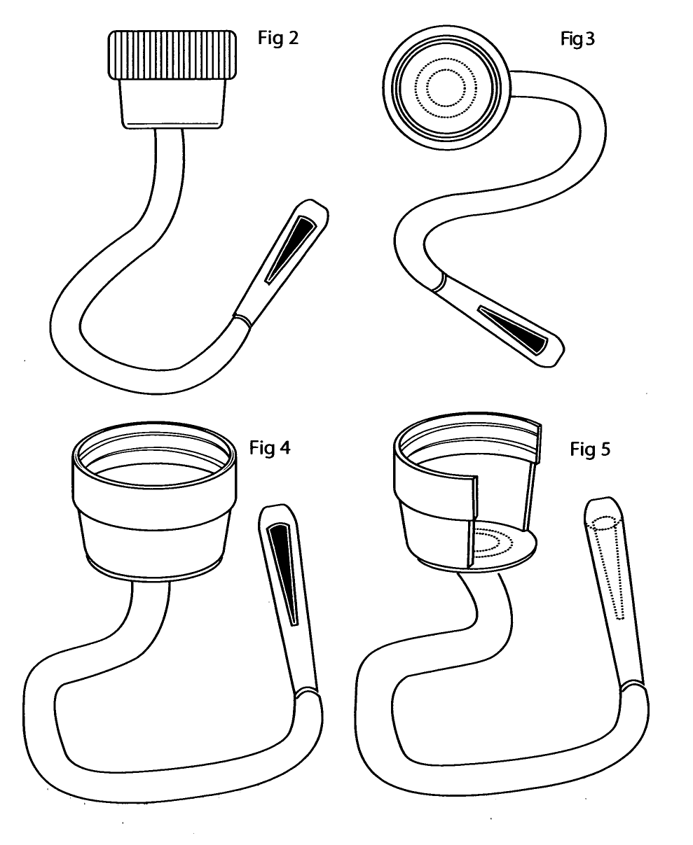

Referring now to the drawings and particularly FIGS. 1, the preferred embodiment of the invention, generally designated FIG. 1-4 is illustrated that the cap 4 or closure assembly communicates to the openings of any standard, common bottled water 6. The syringe 2 comprises a hollow vaginal insert. When the cap 4 of vaginal syringe with hose is completely assembled to the neck 5 of the standard, common bottled water 5, the syringe2 may now be inserted inside the vagina. The bottle 6 can be raised upwardly to allow the water flow naturally down to the cap 4, to the hose 3, to the syringe 2, then to the ejection hole 1. In addition, when standard, common bottled water 6 is compressed by hand, the water due to pressure flows from standard, common bottled water 6, to the cap 4, to the hose 3, to the syringe 2 then flows both to the ejection hole 1. Suitable pressure may then be applied to provide the desired flow rate of water until the standard, common bottled water 6 is empty. The syringe 2 may then be removed and disposed.

As describe above, the vaginal syringe with hose is only one piece which has a cap that communicates to the openings of any standard, common bottled water. When the vaginal syringe with hose is completely assembled to the neck of the standard, common bottled water, insert the syringe into vagina. The standard, common bottle can be raised upwardly to allow the water flow naturally down to the cap, to the hose, to the syringe, then to the ejection hole. In addition, when standard, common bottled water is compressed by hand the water flows down to the cap, then to the hose, then to the syringe, then to the ejection holes. The Vaginal syringe with hose may then be removed and disposed

While a full and complete description of the invention has been set forth in accordance with the dictates of the Patent Statutes, it is to be understood that the invention is not intended to be limited to the specific form herein shown.

Accordingly, modification of the invention may be shown without departing from the spirit hereof or the scope of the accompanying claims.

I claim:

1-13. (canceled)

14. A syringe/nozzle with hose for vaginal/ anus irrigation, compromising:
a. The vaginal/anus syringe with hose that has a cap is one piece that communicates/ seals on the opening of all standard bottled water available in the market
b. The Syringe is connected to the tube. The tube also has internal axial passage through which the fluid will pass from the bottled water to the syringe
c. The tube is flexible and can be manufactured in any other material
d. The syringe/nozzle syringe with hose can be manufactured in plastic like polyethylene; however, it can also be manufactured in any other type of material.
e. The Thread diameter may vary in thickness and in height.
f. The syringe/nozzle can be use for vagina or anus

15. Washer can be added inside the cap to prevent leaking.
